(19) United States
(12) Patent Application Publication
(10) Pub. No.: US 2014/0127752 A1
(43) Pub. Date: May 8, 2014

(54) METHOD, COMPOSITION, AND REAGENT KIT FOR TARGETED GENOMIC ENRICHMENT

(71) Applicants: Zhaohui Zhou, San Ramon, CA (US); Yue Xu, Alamo, CA (US); Qun Shan, Albany, CA (US)

(72) Inventors: Zhaohui Zhou, San Ramon, CA (US); Yue Xu, Alamo, CA (US); Qun Shan, Albany, CA (US)

(21) Appl. No.: 14/074,679

(22) Filed: Nov. 7, 2013

Related U.S. Application Data
(60) Provisional application No. 61/723,320, filed on Nov. 7, 2012.

(51) Int. Cl. C12N 9/22; C12P 19/34

(52) U.S. Cl. CPC C12N 9/22; C12P 19/34
USPC 435/91.53; 435/196

(57) ABSTRACT

A composition and method of cleaving a target DNA and isolating a DNA sequence of interest, directed by a targeting oligonucleotide (“ON”) including a DNA binding agent (stable or unstable), is disclosed. The targeting ON binds to the target DNA before or during DNA cleavage. After cleavage, the isolation of the DNA fragment of interest is facilitated by the affinity tag on the targeting ON or an affinity tag attached using either ligation or polymerase extension method.

### PRIORITY CLAIM

This application claims the benefit of priority of U.S. Provisional Patent Application Ser. No. 61/723,320 filed Nov. 7, 2012, which is incorporated herein by reference in its entirety.

### FIELD OF THE DISCLOSURE

This invention relates to method, composition, and reagent kit for cleaving and isolating dsDNA fragment with sequence specificity from larger DNA piece or genomic DNA. One of the applications of the invention is for targeted genomic enrichment, for example, to isolate DNA region of interest from whole genome for DNA sequencing.

### BACKGROUND OF THE INVENTION

The advancement in next-generation sequencing technologies has improved our ability to sequence large genomes at a lower cost and faster speed than ever before. However, it is still not feasible to apply whole genome sequencing routinely in clinical settings. The primary reason is that the cost and time of sequencing the entire genome with an accuracy level sufficient to call the variant of interest is still prohibitively high. Contrary to common conception that a person only needs to have his/her gene sequenced once in a lifetime, sequencing may be required multiple times each for a specific purpose. For examples, in cancer diagnostics, heterogeneous cell populations such as tumor cells and normal cells would be sequenced at the same time. In analyzing disease progression, cells from the same source may need to be sequenced at different times. Sequencing may also be applied in prenatal diagnostics to specific cell populations.

In many applications, the goal is only to get an accurate picture of a certain region or regions of the genome of these particular cell populations. Without isolating the specific genomic region, whole genome sequencing is not only wasteful, but also causes delay and inaccuracy. Therefore, a genomic enrichment method that allows isolation of a specific region or regions of interest will lower the cost of sequencing, improve accuracy, and cut time to result significantly.

A number of methods have been used for genome enrichment. One method is PCR based, in which multiple PCR primers are designed and tested. However, PCR amplification and normalization process is labor intensive, and as a result, this method cannot be applied universally. In addition, PCR can only be used for DNA fragments of certain limited size ranges, and complexity of the genome makes it hard to achieve high multiplex PCR with consistent result. A second method is based on sequence specific ligation followed by universal PCR. Again, ligation probe design, process optimization, and size limitation make it less than ideal. A third method is microarray hybridization based. The genomic DNA is sheered into small pieces, and a subset of genomic DNA sequences is captured based on complementary sequence identity. The captured DNA fragments are then taken through the typical library construction protocol.

A common characteristic of the existing targeted genomic enrichment methods is that the DNA region of interest, if more than a few hundred bases long, is captured in small fragments no more than a few hundred bases long. In PCR based methods, the length of each fragment is limited by the ability to reliably and consistently PCR amplify the fragment and is generally a few hundred bases long. In hybridization methods, the genomic DNA is randomly sheared into pieces of about a few hundred bases long, and then each piece is captured through hybridization. There are many inherent problems with capturing a long DNA region of interest in small fragments: (1) not all fragments are captured with the same efficiency, and some fragments may be missed altogether, and (2) many probes will have to be designed and made to cover the entire length of the region of interest, resulting in higher cost. Additionally, PCR may introduce errors to the amplified fragments. For hybridization, the specificity is low, and the processing time is long.

### BRIEF SUMMARY OF THE INVENTION

The key to overcoming the shortcomings of the existing targeted genomic enrichment methods is to be able to cleave and isolate a long DNA region of interest in large fragments, preferably in one whole piece, rather than isolating many short fragments like in current methods. This requires the ability to (1) cleave a target DNA with sequence specificity at predetermined sites, and (2) isolate the cleaved DNA region of interest.

Described herein are method, composition, and reagent kit for cleaving and purifying a DNA fragment of interest 5×10²-1×10⁸-base pairs long, enabling targeted genomic enrichment and selective genomic sequencing with higher specificity, simpler work flow, and lower cost. Central to the invention is an engineered stable-binding sequence specific DNA nuclease that is capable of cleaving the target DNA with sequence specificity and also aiding the isolation of the cleaved DNA fragment through stable-binding. Stable-binding means that one or more components of the engineered nuclease are able to form a stable complex, through covalent bond or non-covalent bond, with the DNA fragment of interest. The stable complex is stable through isolation and thus facilitates the isolation of the fragment of interest, and then the stable complex may be broken up to allow sequencing of the fragment of interest. Sequence specific means that the engineered nuclease is capable of cutting DNA with sequence specificity of eight base pairs or better. A specific sequence must be present for the engineered nuclease to cut. The cutting point may or may not be precisely at any particular base, but will be at close to where it is directed by the targeting sequence. Non-specific background cutting may also be present.

In an embodiment of the invention, a composition includes an engineered stable-binding sequence specific DNA nuclease. The nuclease includes one or more targeting oligonucleotides. The nuclease is capable of cutting a target double stranded DNA with sequence specificity greater than eight base pairs long. The targeting oligonucleotide includes one or more affinity tags; alternatively, an affinity tag is added to target DNA sequences after DNA nuclease cut. Purification of a piece of DNA fragment of interest cut by the sequence specific DNA nuclease is facilitated by the affinity tag.

In another embodiment, a method for cutting out a DNA fragment of interest from a target DNA includes: contacting a target DNA with an engineered stable-binding sequence specific DNA nuclease described above; and isolating the DNA fragment of interest.

In yet another embodiment, a reagent kit includes an engineered sequence specific DNA nuclease, wherein the engineered stable-binding sequence specific DNA nuclease is capable of cutting a target double stranded DNA with sequence specificity greater than eight base pairs long; and the targeting oligonucleotide includes one or more affinity tags or an affinity tag is added to target DNA sequences after DNA nuclease cut. Purification of a piece of DNA fragment of interest cut by the sequence specific DNA nuclease is facilitated by the affinity tag.

### BRIEF DESCRIPTION OF THE DRAWINGS

FIG. 1 is a schematic diagram of sequence specific nuclease that includes RecA and Ref proteins, which is bound to a target DNA.

FIG. 2 is a schematic diagram of a sequence specific nuclease that includes Cas9 protein or variant thereof, which is bound to a target DNA.

FIG. 3 is a schematic diagram of a sequence specific nuclease that includes a chemical nuclease, which is bound to a target DNA.

### DETAILED DESCRIPTION OF THE INVENTION

In an embodiment of the invention, an engineered stable-binding sequence specific DNA nuclease includes a targeting oligonucleotide (“ON”) that is homologous to a selected binding site on a target double stranded DNA (“dsDNA”). Homologous means that the targeting ON is complementary to one strand on the target dsDNA, and is thus capable of forming a triple helix with that target dsDNA, or forming a double helix with the complementary strand. The targeting ON includes a stable-binding agent, which can be a DNA crosslinking agent, a minor groove binder, an intercalator or another agent that stabilizes the target DNA-targeting ON chimera, and the targeting ON further includes one or more affinity tags or is bound to a solid support.

The engineered stable-binding sequence specific DNA nuclease binds to a target DNA at the binding site, forming a target DNA-engineered stable-binding sequence specific DNA nuclease complex. The targeting ON binds to the target DNA, and the engineered stable-binding sequence specific DNA nuclease cuts the target DNA at a cleavage point that is on or near the binding site. After the target DNA is cut, the DNA fragment of interest, which is cross-linked or otherwise stably bound to the targeting ON, is isolated, aided by the affinity tag or solid support on the targeting ON.

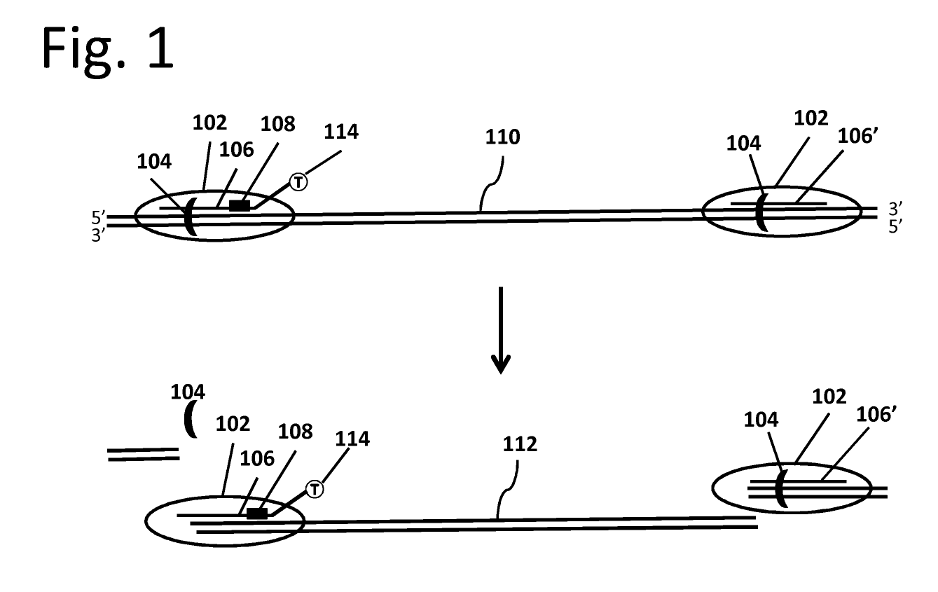

In a first aspect of the embodiment as shown in FIG. 1, the engineered stable-binding sequence specific DNA nuclease includes RecA 102 and Ref 104 proteins and variants thereof, and targeting ON 106 and 106′. Other ingredients of a scission reaction may include ATP and Mg²⁺. The use of RecA, Ref protein, and a targeting ON as a programmable sequence specific DNA nuclease has been described in U.S. patent application Ser. No. 13/208,985 by Cox et al. (the “Cox Application”), a journal article M. C. Gruenig et al. Journal of Biological Chemistry 2011, 286(10), 8240-8251 (the “Cox Article”), and numerous other references. The Cox Application and Cox Article are incorporated herein by reference. The difference is that the targeting ON 106 here includes stable-binding agent 108, and also includes affinity tag 114 or is linked to a solid support.

The RecA protein can be an E. Coli (strain K12) RecA protein (Uniprotsp POA7G6) or a mutant thereof as described in the Cox Application. A RecA variant is defined broadly to include a RecA homolog derived from a common ancestor that performs the same function as RecA in other bacterial species or related families. Non-limiting examples of RecA homologs known in the art include RecA proteins from Deinococcus radiodurans, the RecA protein from Pseudomonas aeruginosa, and the RecA protein derived from Neisseria gonorrhoeae. A RecA variant is also defined to include a polypeptide having at least 40% sequence identity to E. Coli (strain K12) RecA protein and retains the RecA functionality. Preferably, the sequence identity is at least 90%, and more preferably, at least 98% sequence identity.

The Ref protein can be an Enterobacteria phage P1 Ref protein (Uniprotsp 35926) as described in the Cox Application. A Ref variant is defined broadly to include Ref homologs derived from common bacteriophage ancestors that perform the same function as Ref in other bacteriophage or bacterial species. Non-limiting examples of Ref homologs include the Enterobacteria phage φW39 recombination enhancement function (Ref) protein, the Enterobacteria phage P7 Ref protein, the recombination enhancement function (Ref) protein of Salmonella entrica subsp. Entericaserovar Newport str. SL317, and the putative phage recombination protein of Bordetella avium str. 197N. A Ref variant is also defined to include polypeptide variants having at least 75% sequence identity to the Enterobacteria phage P1 Ref protein (Uniprotsp 35926) and retains the Ref functionality. Preferably, the sequence identity is at least 90% to the reference sequence; more preferably, it is at least 98%.

Special RecA and Ref protein variants can be made to optimize the cutting efficiency, binding affinity before and after cutting, and/or sequence specificity. RecA-Ref fused protein variants can also be prepared through standard procedures, and screened for the desired properties.

The targeting ONs 106 and 106′ can be single-stranded DNA, RNA, LNA, PNA, or other DNA analogs, which may include phosphorothioate-DNA in which the phosphothiodiesters take place of the usual phosphodiesters, phosphorothioate-RNA, DNA in which thymidine is substituted with uridine, DNA in which guanidine is substituted with inosine. The DNA analog may include modified deoxyriboses, modified nucleobases, and modified phosphodiesters, which modifications may be currently known in the literature, for example, the DNA analogs described by Aboul-Fadl, Current Medicinal Chemistry, 12, 763-771 (2005), which is incorporated herein by reference, or later developed as long as the DNA analog is capable of sequence specific Watson-Crick base pairing with a complementary DNA.

The targeting ON 106 or 106′ includes a targeting sequence that is 30-200 nucleotides long complementary to one of the strands on the intended biding site. Preferably, the targeting sequence is 50-150 nucleotides long. The entire targeting ON may be 30-3000 nucleotides long.

The targeting ON 106 includes one or more stable-binding agents 108. The stable-binding agent 108 can be anywhere on the targeting ON as long as it does not interfere with RecA/Ref mediated target DNA recognition and cleavage. In some variants, the stable-binding agent is a cross-linking agent that forms a cross-linkage between the targeting ON and the fragment of interest, not on the unwanted sequence of the target DNA 110 that needs to be removed. The cross-linking agents can be a psoralen (see for example, S. Cheng et al., J. Biol. Chem. 1988, 263(29), 15110-7; F. Nagatsugi and S. Imoto, Org. Biomol. Chem. 2011, 9, 2579-85); furan derivatives (stable-binding triggered by singlet oxygen, see M. O. de Beeck and A. Madder, J. Am. Chem. Soc. 2012, 134, 10737-40), 3-cyanovinylcarbazole derivatives (ultrafast reversible photo-crosslinking, 1 second to a few minutes, stable-binding with 366 nm light and reversal with 312 nm light, see Y. Yoshimura and K. Fujimoto, Org. Lett. 2008, 10(15), 3227-30); ruthenium complexes, phenylselenyl compounds under oxidative condition or photo-irradiation, 2-amino-6-vinylpurine (2-AVP) derivatives, and 4-amino-6-oxo-2-vinylpyrimidine (4-AOVPY) derivatives (see review, F. Nagatsugi and S. Imoto, Org. Biomol. Chem. 2011, 9, 2579-85); and thionucleosides (see B. Skalski et al., J. Org. Chem. 2010, 75, 621-6; K. Onizuka et al., Bioconjugate Chem. 2009, 20, 799-803; and L. Lindqvist et al., RNA, 2008, 14, 960-9). In some other variants, the stable-binding agent 108 is a minor groove binder. In further variants, the stable-binding agent 108 is an intercalator.

The targeting ON 106 may include an affinity tag 114 or is bound to a solid support (not shown). The affinity tag 114 may be captured on a solid support, facilitating the DNA fragment-targeting ON chimera to be isolated. The affinity tag may be biotin that can be recognized by avidin. The affinity tag may include multiple biotin residues for increased binding to multiple avidin molecules. The affinity tag may include a functional group such as an azido group or an acetylene group, which enables capture through copper(I) mediated click chemistry (see H. C. Kolb and K. B. Sharpless, Drug Discovery Today, 2003, 8(24), 1128-1137). In some other variations, the affinity tag may include an antigen that may be captured by an antibody bound on a solid support. Other examples of affinity tag include, but not limited to, HIS-tag, Calmodulin-tag, CBP, CYD (covalent yet dissociable NorpD peptide), Strep II, FLAG-tag, HA-tag, Myc-tag, S-tag, SBP-tag, Softag-1, Softag-3, V5-tag, Xpress-tag, Isopeptag, SpyTag, B, HPC (heavy chain of protein C) peptide tags, GST, MBP, biotin carboxyl carrier protein, glutathione-S-transferase-tag, green fluorescent protein-tag, maltose binding protein-tag, Nus-tag, Strep-tag, and thioredoxin-tag.

In other variations, the targeting ON is bound to a solid support. In this case, the binding complex is formed on a solid support, the DNA scission process occurs on the solid support, and after scission, the binding complex including the DNA fragment of interest 112 remains bound on the solid support. The solid support may be glass, plastic, porcelain, resin, sepharose, silica, or other material. The solid support may be a plate that is substantially flat substrates, gel, microbeads, magnetic beads, membrane, or other suitable shape and size. The microbeads may have diameter between 10 nm to several millimeters. The solid support may be non-porous or porous with various density and size of pores. With the DNA fragment 112 captured on a solid support, unwanted DNA may be washed away. Then the DNA fragment will be released from the solid support, for example, by using restriction enzyme, by cleavage of the cross-link between the DNA fragment of interest and the targeting ON if the cross-link is a reversible one, by cleaving the link between the targeting ON and the solid support if that link is designed to be cleavable.

In variants where the stable-binding agent is a cross-linking agent, the target DNA 110 is cross-linked to the targeting ON under appropriate conditions. When the cross-linking agent is a psoralen, 3-cyanovinylcarbazole, ruthenium complex, or phenylselenyl, stable-binding occurs with UV irradiation at the respective wavelength. When the cross-linking agent is furan, phenylselenyl, thionucleosides, appropriate oxidative conditions are applied to cross-link. The cross-linking agent 2-AVP cross-links at neutral condition (faster at pH 5), and 4-AOVPY cross-links with about 60-70% yield at pH 7 in 1.5 hr, both without the need for light, oxidation or other extraneous coupling conditions.

The target DNA 110 is cleaved by incubating with the RecA, Ref, or variants thereof, the targeting oligonucleotide, ATP, and Mg²⁺ in a suitable buffer at a suitable temperature for a suitable length of time. The order of adding the foregoing reagents can be in any order. In a preferred embodiment, first the RecA and targeting ON are incubated in a buffer with ATP, Mg²⁺, and an ATP regeneration system. The target DNA 110 is added next, and a cross-linking condition is applied where the stable-binding agent is a cross-linking agent. Then the Ref is added, and the solution is incubated at 37° C. for 3 hours before taken up for further treatment. Further details of the reaction condition containing a single targeting oligonucleotide can be found in the Cox application, which is incorporated by reference.

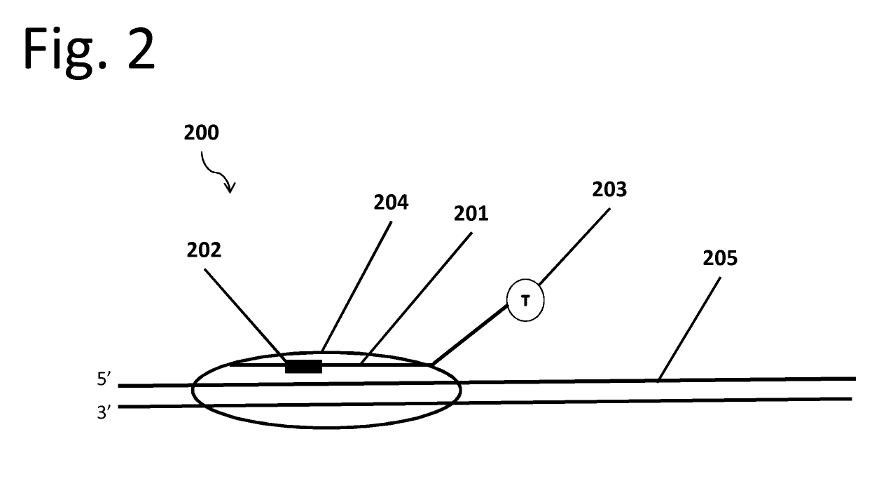

In a second aspect of the embodiment as shown in FIG. 2, the sequence specific stable-binding DNA nuclease 200 includes a targeting ON 201 that is a RNA or analogs thereof that may or may not include a stable-binding agent 202. The nuclease 200 includes an affinity tag 203 or is linked to a solid support (not shown). The nuclease 200 further includes clustered regularly interspaced short palindromic repeats (CRISPR) associated Cas9 protein or variant thereof 204. The targeting ON 201 directs the Cas9 protein or variant thereof 204 to introduce double-stranded breaks in target DNA 205. The use of Cas9 protein 204 as a programmable sequence specific DNA endonuclease is described in M. Jinek et al. Science 2012, 337, 816-821, which in incorporated herein by reference.

The Cas9 protein 204 can be derived from the pathogen Streptococcus pyogenes as described by M. Jinet et al. (id.) A Cas9 variant is defined broadly to include a mutant of Cas9 that maintain part or all of Cas9 functions, or a Cas9 homolog derived from a common ancestor that performs the same or similar function as Cas9 in other bacterial species or related families. Cas9 protein can be of type I, II, or III. A Cas9 variant is also defined to include a peptide having at least 40% sequence identity to Streptococcus pyogenes Cas9 protein and retains the Cas9 functionality. Preferably, the sequence identity is at least 90%, and more preferably, at least 98% sequence identity.

The targeting ON 201 includes a target DNA recognition sequence at the 5′-end that is homologous to a binding site on the double stranded target DNA 205. The targeting ON may include an internal hairpin structure downstream to the target recognition sequence, but another oligonucleotide having a sequence complementary to a sequence downstream to the target recognition sequence may be added to form a duplex, which is required for programming the Cas9 protein to cut the target DNA. A PAM sequence having a GG dinucleotide adjacent to the targeted sequence is required on the target DNA for the wild-type Cas9 protein to function, but a Cas9 variant may be engineered to eliminate the GG sequence requirement. The targeting ON 201 can be a single-stranded RNA, DNA. LNA, PNA, other RNA analogs including phosphorothioate RNA, phosphorothioate DNA, RNA or DNA with modified nucleobases, modified phosphodiesters, modified ribose or deoxyribose, or combinations thereof. For examples, see Aboul-Fadl, Current Medicinal Chemistry, 2005, 12, 763-771, which is incorporated herein by reference in its entirety. Preferably, the targeting ON is a RNA or RNA analog.

The stable-binding agent 202 can be anywhere on the targeting ON 201 that is effective for stable-binding to the target DNA yet does not interfere with target recognition and cleavage. In some variants, the stable-binding agent 202 is a cross-linking agent, which forms a cross-linkage that must be located between the targeting ON and the region of interest, rather than on a unwanted side of the target DNA that needs to be removed. The stable-binding agent can be a psoralen (see for example, S. Cheng et al., J. Biol. Chem. 1988, 263(29), 15110-7; F. Nagatsugi and S. Imoto, Org. Biomol. Chem. 2011, 9, 2579-85); furan derivatives (stable-binding triggered by singlet oxygen, see M. O. de Beeck and A. Madder, J. Am. Chem. Soc. 2012, 134, 10737-40), 3-cyanovinylcarbazole derivatives (ultrafast reversible photo-crosslinking in 1 second to a few minutes, stable-binding with 366 nm light and reversal with 312 nm light, see Y. Yoshimura and K. Fujimoto, Org. Lett. 2008, 10(15), 3227-30); ruthenium complexes, phenylselenyl compounds under oxidative condition or photo-irradiation, 2-amino-6-vinylpurine (2-AVP) derivatives, and 4-amino-6-oxo-2-vinylpyrimidine (4-AOVPY) derivatives (see review, F. Nagatsugi and S. Imoto, Org. Biomol. Chem. 2011, 9, 2579-85); or thionucleosides (see B. Skalski et al., J. Org. Chem. 2010, 75, 621-6, K. Onizuka et al., Bioconjugate Chem. 2009, 20, 799-803, L. Lindqvist et al., RNA, 2008, 14, 960-9). In some other variants, the stable-binding agent 202 is a minor groove binder. In further variants, the stable-binding agent 202 is an intercalator.

The targeting ON may include one or more affinity tags 203 or be linked to a solid support. The affinity tag 203 can be anywhere on the targeting ON 201 provided that it does not interfere with Cas9 mediated target DNA recognition and cleavage and that it is at a position effective for isolating the targeting ON-fragment of interest chimera after cleavage. The affinity tag may be biotin that will be recognized by avidin. The affinity tag may include multiple biotin residues for increased binding to multiple avidin molecules. The affinity tag may include a functional group such as an azido group or an acetylene group, which enables capture through copper(I) mediated click chemistry (see H. C. Kolb and K. B. Sharpless, Drug Discovery Today, 2003, 8(24), 1128-1137). In some other variations, the affinity tag may include an antigen that may be captured by an antibody bound on a solid support. Other examples of affinity tag include, but not limited to, HIS-tag, Calmodulin-tag, CBP, CYD (covalent yet dissociable NorpD peptide), Strep II, FLAG-tag, HA-tag, Myc-tag, S-tag, SBP-tag, Softag-1, Softag-3, V5-tag, Xpress-tag, Isopeptag, SpyTag, B, HPC (heavy chain of protein C) peptide tags, GST, MBP, biotin carboxyl carrier protein, glutathione-S-transferase-tag, green fluorescent protein-tag, maltose binding protein-tag, Nus-tag, Strep-tag, and thioredoxin-tag.

In another variation, the targeting ON 201 is bound to a solid support. In this case, the binding complex is formed on a solid support, the DNA stable-binding and scission processes occur on the solid support, and after scission, the binding complex including the DNA fragment of interest remains bound on the solid support. The solid support may be glass, plastic, porcelain, resin, sepharose, silica, or other material. The solid support may be a plate that is substantially flat substrates, gel, microbeads, magnetic beads, membrane, or other suitable shape and size. The microbeads may have diameter between 10 nm to several millimeters. The solid support may be non-porous or porous with various density and size of pores. With the DNA fragment captured on a solid support, unwanted DNA may be washed away. Then the DNA fragment of interest will be released from the solid support, for example, by using restriction enzyme, by cleavage of the cross-link between the DNA fragment of interest and the targeting ON if the cross-link is a reversible one, or by cleaving the link between the targeting ON and the solid support if that link is designed to be cleavable.

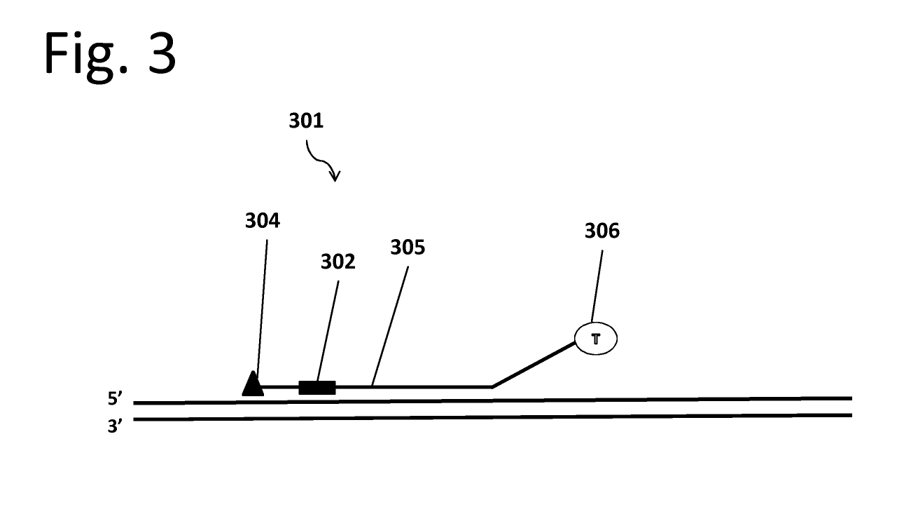

In a third aspect of the embodiment as shown in FIG. 3, the engineered sequence specific DNA nuclease is a sequence specific chemical nuclease 301, which includes a chemical nuclease 304 linked to a sequence specific DNA binder 305, and optionally a stable-binding agent 302. The nuclease 301 further includes an affinity tag 306 or is attached to a solid support (not shown). The sequence specific DNA binder can be an oligonucleotide, an engineered Zinc-finger protein, a transcription activator like effector (TALE), or a DNA binding chemical substance such as distamycin. The oligonucleotide can be an oligodeoxyribonucleotide, oligoribonucleotide, or analogs thereof including phosphorothioate, zip nucleic acids, and other DNA or RNA analogs, for example, the DNA analogs described by Aboul-Fadl, Current Medicinal Chemistry, 2005, 12, 763-771, which is incorporated herein by reference. The chemical nuclease can be any chemical reagent that cleaves DNA, such as 1,10-phenanthroline-copper and derivatives thereof (Sigman et al. Chem. Rev. 1993, 93, 2295-2316; Kellett et al. Med. Chem. Comm. 2011, 2, 579; Chakravarty et al. Proc. Indian Acad. Sci. 2002, 114(4) 391-401), EDTA-Fe (Schultz and Dervan, J. Am. Chem. Soc. 105, 7748-7750, 1983). The double-strand DNA cleaving activities of the chemical nucleases are well known in the literature, including the references cited above, which are incorporated herein by reference. The stable-binding agent 302 can be a cross-linking agent, a minor groove binder, or an intercalator, as described above.

A variant of the above described engineered stable-binding sequence specific DNA nuclease is non-stable-binding sequence specific DNA nuclease, which is similar but without the stable-binding agent on the targeting ON. Engineered sequence specific DNA nucleases without stable-binders employing RecA/Ref, TALEN, and chemical nuclease have been previously described in a provisional patent application, Ser. No. 61/679,725 filed Aug. 5, 2012, which is incorporated herein by reference in its entirety. The non-stable-binding engineered sequence specific DNA nuclease employing Cas9 or other members of the CRISPR-Cas system (Jinek, et al. Science 337 (6096): 816-21; Cong, et al. Science 339 (6121): 819-23; Mali, et al. Science 339 (6121): 823-6) is similar to the stable-binding version, with the main difference being that the targeting ON is without a stable-binding agent. Where only non-stable-binding sequence specific DNA nuclease is used, the DNA fragment of interest may be selectively attached to one or more affinity tags or a solid support, using DNA ligase, polymerase, or other suitable reagents.

In another aspect of the embodiment, an engineered stable-binding sequence specific DNA nuclease may be used in combination with a non-stable-binding engineered sequence specific DNA nuclease in cleaving a fragment of interest. Because only one stable-binding sequence specific DNA nuclease, hence only one targeting ON with cross-linker, is used, only one targeting ON is stably bound to a fragment of interest.

In yet another aspect of the embodiment, multiple pairs of engineered stable-binding sequence specific DNA nucleases are employed in one reaction. Thus, multiple DNA fragments covering multiple regions of DNA sequences of interest may be cut and isolated in one run. In some applications, if the DNA sequence of interest is located near the end of the target DNA, then only one engineered stable-binding sequence specific DNA nuclease is required for cutting and isolating the DNA fragment.

In another aspect of the embodiment, multiple pairs of engineered stable-binding sequence specific DNA nucleases are employed in one reaction to cut out the same DNA sequence of interest from the same target DNA, but at different cutting points, resulting in multiple fragments of interest all including the same DNA region of interest. By carrying out such redundant cuts for the same DNA sequence of interest, the overall efficiency, i.e. percentage of target DNA cut, may be increased. By combining the forgoing two embodiments, multiple DNA fragments covering the same DNA sequence of interest, as well as multiple DNA sequences of interest, may be cut and isolated in one run.

While embodiments and applications of this disclosure have been shown and described, it would be apparent to those skilled in the art that many more modifications and improvements than mentioned above are possible without departing from the inventive concepts herein. The disclosure, therefore, is not to be restricted except in the spirit of the appended claims.

What is claimed is:

1. A composition comprising an engineered stable-binding sequence specific DNA nuclease, comprising
one or more targeting oligonucleotides, wherein:
said engineered sequence specific DNA nuclease is capable of cutting a target double stranded DNA with sequence specificity greater than eight base pairs long;
said targeting oligonucleotide includes one or more affinity tags ; and
purification of a piece of DNA fragment of interest cut by said sequence specific DNA nuclease is facilitated by said affinity tag.

2. The composition of claim 1, wherein said engineered sequence specific DNA nuclease comprises:
a Cas9 protein or a variant thereof;
a targeting oligonucleotide;
wherein one of the above components includes an affinity tag or is bound to a solid support.

3. The composition of claim 2, comprising a Cas 9 protein.

4. The composition of claim 2, comprising a targeting DNA that is a single-stranded DNA or RNA 30-1,000 nucleotides in length, wherein a 30-150 nucleotides long sequence is complementary to a sequence on one strand of the target double stranded DNA.

5. The composition of claim 2, wherein said affinity tag is selected from the group consisting of biotin, azido group, acetylene group, HIS-tag, Calmodulin-tag, CBP, CYD, Strep II, FLAG-tag, HA-tag, Myc-tag, S-tag, SBP-tag, Softag-1, Softag-3, V5-tag, Xpress-tag, Isopeptag, SpyTag, B, HPC peptide tags, GST, MBP, biotin carboxyl carrier protein, glutathione-S-transferase-tag, green fluorescent protein-tag, maltose binding protein-tag, Nus-tag, Strep-tag, and thioredoxin-tag.

6. The composition of claim 2, wherein said solid support is a plate, membrane, gel, magnetic bead, or microbead.

7. A method for cutting out a DNA fragment of interest from a target double stranded DNA, comprising:
contacting said target double stranded DNA with a composition comprising an engineered sequence specific DNA nuclease that includes one or more targeting oligonucleotides, wherein said engineered sequence specific DNA nuclease is capable of cutting a double stranded DNA with sequence specificity greater than eight base pairs long; and
isolating the DNA fragment of interest.

8. The method of claim 7, wherein:
said composition includes an engineered stable-binding sequence specific DNA nuclease, and one or more components of the engineered stable-binding sequence specific DNA nuclease include one or more affinity tags or are bound to a solid support; and
purification of said DNA fragment of interest is facilitated by said affinity tag or solid support.

9. The method of claim 7, further comprising:
attaching one or more affinity tags to said DNA fragment of interest; and
isolating said DNA fragment of interest facilitated by said affinity tag.

10. The method of claim 7, wherein said composition includes one or more targeting oligonucleotides, and the composition causes the target DNA to be cut by said engineered sequence specific DNA nuclease at only one end of the DNA fragment of interest in such a way that an affinity tag can be attached to the cut DNA fragment and this DNA fragment can be isolated using said affinity tag.

11. The method of claim 8, wherein said composition includes at least one pair of targeting oligonucleotides, and the composition causes the target DNA to be cut by said engineered stable-binding sequence specific DNA nuclease at both ends of the DNA fragment of interest in such a way that at least one pair of targeting oligonucleotides remain bound to the DNA fragment of interest after the target DNA is cut.

12. The method of claim 8, wherein said composition includes at least one pair of targeting oligonucleotides, and the composition causes the target DNA to be cut by said engineered stable-binding sequence specific DNA nuclease at both ends of the DNA fragment of interest in such a way that only one targeting oligonucleotide remains bound to the DNA fragment of interest after the target DNA is cut, and wherein said targeting oligonucleotide that remains bound includes said affinity tag or is bound to said solid support.

13. A reagent kit comprising an engineered stable-binding sequence specific DNA nuclease, wherein:
said engineered stable-binding sequence specific DNA nuclease is capable of cutting a target double stranded DNA with sequence specificity greater than eight base pairs long;
said engineered stable-binding sequence specific DNA nuclease includes one or more targeting oligonucleotides that includes one or more stable-binding agent;
at least one component of the engineered stable-binding sequence specific DNA nuclease includes an affinity tag or is bound to a solid support; and
purification of a piece of DNA fragment of interest cut by said engineered stable-binding sequence specific DNA nuclease is facilitated by said affinity tag or solid support.

14. The reagent kit of claim 13, wherein said engineered stable-binding sequence specific DNA nuclease comprises a Cas9 protein or a variant thereof.

15. The reagent kit of claim 13, comprising a Cas 9 protein.

16. The reagent kit of claim 13, comprising a targeting DNA that is a single-stranded DNA or RNA 30-1,000 nucleotides in length, wherein a 30-150 nucleotides long sequence is substantially complementary to a sequence on one strand of the target double stranded DNA.

17. The reagent kit of claim 13, comprising a Cas 9 mutant protein.

18. The reagent kit of claim 13, wherein said affinity tag is selected from the group consisting of biotin, azido group, acetylene group, HIS-tag, Calmodulin-tag, CBP, CYD, Strep II, FLAG-tag, HA-tag, Myc-tag, S-tag, SBP-tag, Softag-1, Softag-3, V5-tag, Xpress-tag, Isopeptag, SpyTag, B, HPC peptide tags, GST, MBP, biotin carboxyl carrier protein, glutathione-S-transferase-tag, green fluorescent protein-tag, maltose binding protein-tag, Nus-tag, Strep-tag, and thioredoxin-tag.

19. The reagent kit of claim 13, wherein said solid support is a plate, membrane, gel, magnetic bead, or microbead.
